(19) United States
(12) Patent Application Publication
(10) Pub. No.: US 2009/0171251 A1
(43) Pub. Date: Jul. 2, 2009

(54) ULTRASOUND TREATMENT OF ADIPOSE TISSUE WITH VACUUM FEATURE

(76) Inventors: Andrey Rybyanets, Yoqneam (IL); Igor Nudelman, Herzliya (IL)

Correspondence Address: EMPK & Shiloh, LLP;c/o Landon IP, Inc., 1700 Diagonal Road, Suite 450, Alexandria, VA, 22314, US

(21) Appl. No.: 12/027,342

(22) Filed: Feb. 7, 2008

Related U.S. Application Data
(63) Continuation-in-part of application No. 12/005,322, filed on Dec. 27, 2007.

(51) Int. Cl. A61N 7/00

(52) U.S. Cl. 601/2

(57) ABSTRACT

There is provided herein, an apparatus for lysing of adipose tissue comprising a transducer adapted to transmit ultrasound to a target area tissue of a subject body; and a vacuum system adapted to suck a surface of the target area tissue into the transducer to facilitate acoustic contact between the surface of the target area tissue and the transducer.

### RELATED APPLICATIONS

This application is a continuation in part of U.S. patent application Ser. No. 12/005,322, filed Dec. 27, 2007, which is hereby incorporated by reference in its entirety.

### FIELD

Methods and apparatus for performing acoustic procedures on tissue and, more particularly, for the controlled ultrasound treatment of adipose tissue, such as for treatment of fat deformities or excesses by lysing adipose (fat) tissue.

### BACKGROUND

The aesthetic medical field is a fast growing area in which medical procedures as well as medical devices are used to promote aesthetic traits. One of the most popular areas in the aesthetic medical field is the removal and/or reduction of subcutaneous fat cell number(s) and adipose tissue volume. Removal and/or reduction of subcutaneous fat cell number(s) and adipose tissue volume may result in the reshaping of body parts, frequently referred to as “body contouring”. To date, various techniques have been proposed to aid in the task of lowering the number and/or volume of fat cells and adipose tissue (such as liposuction, Ultrasound Assisted Lipoplasty (UAL), medications, ointments, laser based procedures, RF based procedures, Ultra Sound based procedures, and the like).

Adipose tissue or fat is generally loose connective tissue composed primarily of adipocytes. Its main role is to store energy in the form of fat, although it also cushions and insulates the body. Obesity in humans and most animals is generally not dependent on the amount of body weight, but on the amount of body fat—specifically adipose tissue. In humans, adipose tissue is located beneath the skin and is also found around internal organs. Adipose tissue is found in specific locations, which are referred to as adipose depots. Adipose tissue contains several cell types, with the highest percentage of cells being adipocytes, which contain fat droplets. Other cell types include fibroblasts, macrophages and endothelial cells. Adipose tissue usually contains many small blood vessels. In the integumentary system, which includes the skin, it accumulates in the deepest level, the subcutaneous layer, providing insulation from heat and cold. Around organs, it provides protective padding. However, its main function is to be a reserve of lipids, which can be burned to meet the energy needs of the body. Adipose depots in different parts of the body have different biochemical profiles. Cellulite is also often included within the scope of fat or adipose tissue.

A widely used technique for lowering the number and/or volume of fat cells (“fat removal technique”) is liposuction. Liposuction is a medical procedure that involves surgical removal of all or part of the subcutaneous fat cell layer in target areas of the body. This procedure is invasive and involves local or general anesthesia. The procedure involves the insertion of, for example, a cannula through a small skin incision into the adipose tissue, whereby the fat is then suctioned out. The cannula may be moved back and forth in different tissue levels covering the volume to be suctioned. The fat is torn and evacuated at the same time. This procedure may require several incisions to be made to the skin and is non-selective, as along with fat tissues, other surrounding tissues, such as blood vessels, nerves and connective tissues may tear. Side effects of this procedure are hematomas, hypo-sensation and pain, and recovery time may be prolonged. Ultrasonically vibrating cannulas have also been used to increase tearing of tissue, which contacts the cannula.

Various other fat removal techniques and procedures have been described, such as, for example, use of medications, ointments, laser based procedures, radio frequency (RF) based procedures, ultrasound based procedures and the like. Ultrasound (or ultrasonic sound) is defined as sound of frequencies too high to be audible by the human ear, such as above approximately 20 kHz (kilo-Hertz). Some sound that is ultrasound to the human ear can be heard by animals, such as dogs. Ultrasound has industrial and medical applications, such as an alternative to X-rays for imaging.

Among the ultrasound based procedures for fat and adipose tissue removal, an additional body contouring solution involves a non-invasive treatment. The non-invasive treatment is based on the application of focused therapeutic ultrasound that selectively targets and disrupts fat cells without substantially damaging neighboring structures. This may be achieved by, for example, a device, such as an ultrasonic transducer, that delivers focused ultrasound energy to the subcutaneous fat layer. Specific, pre-set ultrasound parameters ensure that the fat cells within the treatment area are targeted and that neighboring structures such as blood vessels, nerves and connective tissue remain essentially intact. Since ultrasonic energy may considerably be attenuated in air, in order to efficiently transmit ultrasonic energy to subcutaneous fat layer, an interposer substance having appropriate acoustic impedance may be placed between the subject body and the device that delivers the focused ultrasound energy. Non-selective or less selective targeting may also be used, which may damage or lyse multiple tissue types in a target area—usually by setting different ultrasound parameters.

The ultrasonic transducer transmits energy either in a continuous wave mode, or in pulses. In a continuous wave mode, there is no cessation in the flow of transmitted energy, and an increase in temperature is therefore usually inevitable. In the pulsed mode, the energy is delivered in bursts, which can be controlled to produce a controlled lower temperature rise. The energy emitted from the ultrasonic transducer can be used for the removal, damage, lysing and/or reduction of subcutaneous fat cell number(s) and adipose tissue volume, which results in reshaping a body (“body contouring”). Currently, the physician contacts the transducer assembly to a subject's skin, such as around the stomach area, and slides it along the skin surface. This concept is disclosed, for example, in U.S. Pat. No. 6,607,498.

Other methods and apparatus can be used for producing lysis of adipose tissue underlying the skin of a subject as by applying an ultrasonic transducer to the subject's skin to transmit therethrough ultrasonic waves focused on the adipose tissue and electrically actuating the ultrasonic transducer to transmit ultrasonic waves to produce cavitational lysis of the adipose tissue without damaging non-adipose tissue. Such concept is disclosed, for example, in U.S. Pat. No. 6,607,498.

A tissue can be non-invasively exposed to ultrasonic energy in a focused or a non-focused manner. When a non-focused transducer is used, all tissue between the transducer, and up to a certain fading distance where energy levels are lower than the therapeutic or effective threshold, are exposed to the ultrasonic energy. When focused ultrasound is used, only the tissue at the focal range or target area/zone of the transducer is specifically affected while other tissues, between the transducer and the focus or beyond, are spared.

Generally, tissue destruction may be performed using high-intensity focused ultrasound (HIFU) energy, which can cause tissue damage by two main mechanisms, namely, thermal and mechanical mechanisms. The thermal mechanism includes an increase of temperature (heating) within the treated area, obtained by a direct absorption of ultrasonic energy by the treated tissue. The increased temperature causes damaging processes, such as coagulation, within the tissue. The mechanical mechanism mainly includes streaming, shear forces, tension and cavitation, which is the formation of small bubbles within the tissue. These processes cause fractionation, rupture and/or liquefaction of cells, which in turn results in tissue destruction. Cavitation is a physical phenomenon in which low-pressure bubbles are formed and then tend to collapse in a liquid. Cavitation near cells will damage or destroy many of the cells. The cavitation phenomenon depends on specific tissue characteristics when employed in a biological environment. This enables tissue differentiation for damage or destruction, which means that fat cells can be destroyed (or damaged sufficiently to die soon after), while blood vessels, peripheral nerves, skin, muscle and connective tissue within the ultrasonic focus, as well as neighboring tissues such as listed above outside the focus, will remain intact.

Other destructive mechanisms, such as cell apoptosis, may also directly or indirectly be involved in the non-invasive ultrasonic treatment.

FIG. 1 illustrates a simple design, which is currently known (alternate shape may not be known) for an acoustic transducer apparatus 100, which may be used for lysis of adipose tissue. A principal component of apparatus 100 is an ultrasonic transducer 102. Transducer 102 may have a concave, such as a hemispherical shape (cross-section). An equatorial plane, indicated by dashed line labeled “HP” may be associated with the hemispherical shape of transducer 102, although transducer 102 is illustrated as not being a complete hemisphere (it is a hemispherical segment). Alternatively, alternate transducers may have other shapes (such as conical, half-cylindrical, flat, slightly curved, and so forth) and may be composed of more than one sub-element, like an array (not shown).

A radius of curvature for transducer 102 may be, for example, approximately 60 mm (millimeters). The inside (lower, as viewed) surface of transducer 102 may focus acoustic energy a distance beyond the hemispherical plane HP, into a subject body 110. Acoustic energy generated by transducer 102 is represented by the arrows converging on an area 112 within (below surface 110A of) subject body 110. Area 112 may be located, for example, approximately 2 mm (millimeters) below surface 110A of subject body 110, and may be referred to as “the area being treated”, or “treatment area”. Other depths may be achieved by shape of transducer(s) (focal point/zone) or by operating parameters. Alternatively, such treatment area may have a shape, or volume, such as an oval of approximately one wavelength (short diameter) and two-three wavelengths (long diameter). Alternate shapes of transducers and/or operating parameters may form other shaped treatment areas (such as half cylinder producing rod shapes, flat transducers producing planar shaped treatment areas, and other shapes).

The concavity of the transducer may be sealed with polyurethane, oil maintained within the concavity by a membrane (such as, polyethylene, nylon or any other material) or with any other appropriate material. Any material sealing or filling the concavity of the transducer should have minimal effect on the intensity or characteristics of the signals produced by the transducer, and may be impedance matched to the characteristics of the acoustic energy emitted by the transducer. In use, transducer 102 may be disposed in a housing or casing (not shown) and placed on (in direct contact with) surface 110A of subject body 110, such as the skin of a patient being treated. An ultrasound gel (not shown) may be used, such as by applying to apparatus 100 or to the patient's skin, to enhance acoustic coupling between apparatus 100 and treatment area 112. A lubricant (not shown) may also be used to minimize effects of friction when moving the transducer assembly apparatus over the skin surface. The ultrasound gel and lubricant may be combined, and may be in non-gel format, such as a cream, liquid, suspension, paste or other form.

In use, apparatus 100 may be moved across surface 110A of subject body 110 to treat an overall area larger than a single treatment area 112.

When using ultrasonic (ultrasound) methods for body contouring, an interposer substance having appropriate acoustic impedance is often placed between the subject's body and the device (assembly apparatus) in order to efficiently transmit ultrasonic energy to a subcutaneous fat layer. The interposer may be a lubricant (for example, castor or other oil may be used for this purpose). Nevertheless, there are still problems related to deficiencies in creating an efficient acoustic contact between the tissue being treated and the transducer. Other problems related to the existing technology involve pain during treatment, non-optimal energy focus and other problems.

In addition, combinations of different ultrasonic treatment and suction of body tissue, for the purpose of enhancing a massage effect, are known in the art. For example, US20040260210 discloses a spout, for face and body treatment, which includes a casing, having an inner chamber and a proximal surface, with respect to a portion of the body, for applying a suction treatment to the portion of the body and at least one ultrasound transducer, mounted on the proximal surface, for applying an ultrasound treatment to the portion of the body. Another example may be found in EP1386597 which discloses an ultrasound endomassage device used in massage, slimming and anti-cellulitis treatments including a casing, which houses in its interior an ultrasound transducer, on whose body are a series of passing holes through which suction, produced by a vacuum pump, is exerted, and the casing has a handle on which indicators and an operating switch can be found.

### SUMMARY

A general object of the present invention is to provide an improved technique for producing lysis of adipose tissue in a non-invasive manner.

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other advantages or improvements.

According to the invention, generally, a method and apparatus is provided for improving ultrasound treatment of adipose tissue by HIFU (high intensity focused ultrasound), including one or more of the following objectives:
1. Fixation of target area tissue during treatment;
2. Pre-stressing of target tissue;
3. Pain decrease;
4. Providing an acoustic contact between tissue and ultrasonic transducer;
5. Providing a combination treatment comprising vacuum massage and HIFU and ultrasonically assisted drug delivery;
6. Treatment time decrease;
7. Continuous supply of contact fluid and/or active agents (such as drugs, and referred to hereinafter as “drugs”) into a contact area; and
8. Providing a possibility of disposable membrane using vacuum clamping.

These objectives may be accomplished, generally, by using a vacuum and/or supplying contact fluids and/or drugs, in conjunction with the ultrasound treatment, as described in greater detail hereinbelow.

In some embodiments, the apparatus can be supplemented by a system for providing vacuum pulsations, which may be synchronized with the electric driving circuitry, such that the vacuum pulses only when the HIFU is being produced, in other words, the vacuum pulses are synchronized with ultrasonic pulses.

In some embodiments, the apparatus can be supplemented by an oil ignition pump for contact fluid supply, such that the contact fluid is sucked by the vacuum pump.

The transducer design can also include a vacuum-clamped disposable hygienic membrane, such as a polyethylene or a silicon membrane. These membranes can be vacuum clamped onto the transducer during treatment and replaced after each treatment. In some embodiments, the transducer may include two vacuum systems, one for sucking the body tissue to allow better acoustic contact and the other to fix the hygienic membrane to the transducer.

In some embodiments of the invention, there is provided an apparatus for lysing of adipose tissue comprising a transducer adapted to transmit ultrasound to a target area tissue of a subject body and a vacuum system adapted to suck a surface of the target area tissue into the transducer to facilitate acoustic contact between the surface of the target area tissue and the transducer. The vacuum system may be adapted to operate in a continuous mode. The vacuum system may also be adapted to operate in a pulsed mode. The vacuum system and said transducer may both be adapted to operate in a pulsed mode. The vacuum system may be adapted to operate in a pulsed mode to produce vacuum pulsation and said transducer is adapted to operate in a pulsed mode to produce ultrasonic pulsation, wherein the vacuum pulsation and the ultrasonic pulsation are adapted to be synchronized. The vacuum system may comprise a vacuum pump.

The apparatus may further include a coupling element for enhancing acoustic coupling of the ultrasound to target area tissue of a subject body. The coupling element may include a membrane disposed between the transducer and the surface of the target area tissue of the subject body. The membrane may include an annular lip protruding from a peripheral region of a bottom surface of the membrane, defining an enclosed space between the bottom surface of the membrane and a surface of the subject body. The annular lip may have a substantially hemispherical profile. The annular lip may have a substantially rectangular profile. The annular lip may have a substantially U-shaped profile. The annular lip may have at least one passageway to allow vacuum suction of the surface of the target area tissue into the transducer.

The apparatus may be adapted to receive a replicable hygiene membrane. The apparatus may be adapted to vacuum-clamp a replicable hygiene membrane.

The apparatus may further include a fluid supplying element for supplying a fluid, through the transducer, to a contact area on the surface of the target area tissue of the subject body.

In some embodiments of the invention, there is provided a method for lysing of adipose tissue comprising providing an ultrasonic transducer applying vacuum to suck a surface of a target area tissue of a subject body into the transducer to facilitate acoustic contact between the surface of the target area tissue and the transducer and transmitting ultrasound by the ultrasonic transducer to the target area tissue. Applying vacuum may include applying vacuum in a continuous mode. Applying vacuum may include applying vacuum in a pulsed mode.

Transmitting ultrasound may include transmitting ultrasound in a pulsed mode. Applying vacuum may include applying vacuum in a pulsed mode to produce vacuum pulsation, wherein transmitting ultrasound comprises transmitting ultrasound in a pulsed mode to produce ultrasound pulsation and wherein the vacuum pulsation and the ultrasonic pulsation are adapted to be synchronized.

The method may further include supplying fluid through the transducer, to a contact area on the surface of the target area tissue of the subject body. Supplying fluid may include injecting the fluid. Supplying fluid may include vacuum-sucking the fluid.

The transducer may be capable of producing ultrasonic waves with an intensity sufficient for inducing cavitational lysis of said adipose tissue. The transducer may be capable of producing ultrasonic waves with an intensity sufficient for inducing thermal lysis of said adipose tissue. The transducer may be actuated to transmit ultrasonic waves of sufficient intensity to cause lysis of said adipose tissue essentially without damaging adjacent non-adipose tissue.

In some embodiments of the invention, there is provided a system for lysing of adipose tissue comprising a transducer adapted to transmit ultrasound to a target area tissue of a subject body a vacuum system adapted to suck a surface of the target area tissue into the transducer to facilitate acoustic contact between the surface of the target area tissue and the transducer and a controller adapted to synchronize the actuation of said transducer and said vacuum system.

In addition to the exemplary aspects and embodiments described above, further aspects and embodiments will become apparent by reference to the figures and by study of the following detailed descriptions.

### BRIEF DESCRIPTION OF THE FIGURES

Examples illustrative of embodiments of the invention are described below with reference to figures (FIGs.) attached hereto. In the figures, identical structures, elements or parts that appear in more than one figure are generally labeled with a same numeral in all the figures in which they appear. Dimensions of components and features shown in the figures are generally chosen for convenience and clarity of presentation and are not necessarily shown to scale. The figures are listed below.

FIG. 1 is a side cross-sectional schematic view of an ultrasonic transducer,which may be used for ultrasound treatment of adipose tissue, according to the prior art.

FIG. 2A is a cross-sectional view of an apparatus, at least portions of which may be used for ultrasound treatment of adipose tissue, according to some embodiments of the invention.

FIG. 2B is a perspective, partial view of a membrane component of the apparatus shown in FIG. 2A.

FIG. 3 is a diagram of a system for performing controlled ultrasound treatment of adipose tissue on a subject body, according to some embodiments of the invention.

FIG. 4 is a cross-sectional view of a “vibration component” of an overall acoustic transducer apparatus, including a vacuum feature, for performing ultrasound treatment of adipose tissue, according to some embodiments of the invention.

FIG. 4A is a cross-sectional view of a portion of the “vibration component” shown in FIG. 4, without the vacuum turned on.

FIG. 4B is a cross-sectional view of a portion of the “vibration component” shown in FIG. 4, with the vacuum turned on.

FIG. 4C is a cross-sectional view of a “vibration component” of an overall acoustic transducer apparatus, including a vacuum feature, for performing ultrasound treatment of adipose tissue, according to some embodiments of the invention.

FIG. 5A is a cross-sectional view of a portion of a membrane component of the “vibration component” shown in FIG. 4 (or FIG. 7), according to some embodiments of the invention.

FIG. 5B is a cross-sectional view of a portion of a membrane component of the “vibration component” shown in FIG. 4 (or FIG. 7), according to some embodiments of the invention.

FIG. 5C is a cross-sectional view of a portion of a membrane component of the “vibration component” shown in FIG. 4 (or FIG. 7), according to some embodiments of the invention.

FIG. 6 is a diagram of a system for performing controlled ultrasound treatment of adipose tissue on a subject body, according to some embodiments of the invention.

FIG. 7 is a cross-sectional view of a “vibration component” of an overall acoustic transducer apparatus, including fluid injection, for performing ultrasound treatment of adipose tissue, according to some embodiments of the invention.

FIG. 7A is a cross-sectional view of a “vibration component” of an overall acoustic transducer apparatus, including fluid injection, for performing ultrasound treatment of adipose tissue, according to some embodiments of the invention.

FIG. 8 is a diagram of a system for performing controlled ultrasound treatment of adipose tissue on a subject body, according to some embodiments of the invention.

FIG. 9 is a cross-sectional view of a “vibration component” of an overall acoustic transducer apparatus, including a vacuum feature and fluid injection, for performing ultrasound treatment of adipose tissue, according to some embodiments of the invention.

FIG. 9A is a cross-sectional view of a “vibration component” of an overall acoustic transducer apparatus, including a vacuum feature and fluid injection, for performing ultrasound treatment of adipose tissue, according to some embodiments of the invention.

### DETAILED DESCRIPTION

In the following description, various aspects of the invention will be described. For the purpose of explanation, specific configurations and details are set forth in order to provide a thorough understanding of the invention. However, it will also be apparent to one skilled in the art that the invention may be practiced without specific details being presented herein. Furthermore, well-known features may be omitted or simplified in order not to obscure the invention.

In the description and claims of the application, each of the words “comprise” “include” and “have”, and forms thereof, are not necessarily limited to members in a list with which the words may be associated.

As referred to herein, the term a “subject body” may include all or any part of a subject body, both internally and/or externally. For example, a “subject body” may include an entire body; body part, such as a limb; an organ, such as for example, a liver; a tissue, such as for example, skin tissue, subcutaneous adipose tissue, blood vessel, nerve tissue and the like; cells, such as, for example, fat cells, blood cells, and the like. The term working surface and user body may interchangeably be used.

As referred to herein the terms transducer, transducer unit, transducing unit, therapeutic transducer and vibration delivery system may interchangeably be used.

As referred to herein, the terms acoustic energy, acoustic waves, ultrasound, ultrasonic energy, ultrasonic waves, vibration energy and vibration waves, may interchangeably be used.

Ultrasonic energy may be transmitted either in a continuous wave mode, or in pulses, to a subject body, to effect controlled ultrasound treatment of adipose tissue. In a continuous wave mode, there is no cessation in the flow of transmitted energy, and an increase in temperature is therefore inevitable. In the pulsed mode, the energy is delivered in bursts, which can be controlled to produce a controlled lower temperature rise.

A pulsed wave can be characterized (besides the frequency and amplitude as in the continuous mode) by two parameters: the pulse length, and the pulse repetition period. The pulse length is defined here as the time in which the intensity is above the value needed for sustaining cavitation; between the pulses, the intensity is below that needed to sustain cavitation. Pulse characteristics are discussed in greater detail in the aforementioned U.S. Pat. No. 6,607,498.

Generally, the ultrasonic energy produced by the transponder and directed into the subject body should be focused, directed to a selected point or small area, to enhance its effectiveness, and to avoid injury to adjacent body structures (such as internal organs).

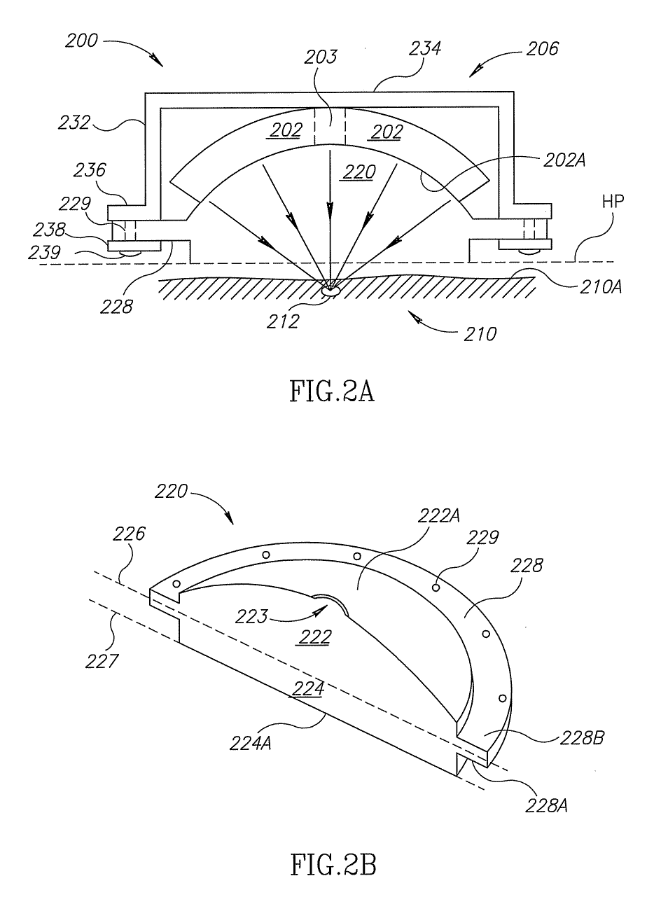

FIG. 2A illustrates a “vibration component” 200 of an overall acoustic transducer apparatus. Main components of the vibration component 200 include an acoustic transducer 202 (compare 102), a membrane 220 and a housing (or casing) 206. The acoustic transducer 202 may optionally include a hole 203 which may be used for placement of the transducer. Membrane 220 is shown in greater detail in FIG. 2B.

A “connection component” (not shown) may be provided to connect transducer 202 to additional devices such as a generator unit that may provide transducer 202 with power, energy, fluids, software instruction, control and feedback, and the like. The vibration component may be used to produce, concentrate and output vibration energy and may come in close proximity and/or contact with a subject body.

In FIG. 2A, a bottom external surface of the vibration component 200, which is a bottom surface 224A of membrane 220, is shown spaced slightly from an external surface 210A of subject body 210 (compare 110), for illustrative clarity. In use, bottom surface 224A of membrane 220 would be in close proximity or contact with surface 210a.

External casing (housing) 206 of vibration component 200 may be constructed of one integral structure or may be constructed of several constituents that are interconnected to form the external casing of vibration component 200.

Acoustic transducer (or “transducing element”) 202 may have a concave, such as a hemispherical shape (cross-section). An inside surface 202A of the transducer is concave and, in use, is oriented towards a subject body 210. An equatorial plane, indicated by dashed line labeled “HP” may be associated with the hemispherical shape of transducer 202, although transducer 202 is illustrated as not being a complete hemisphere. The hemispherical plane “HP” may be substantially coincident with bottom surface 224A of membrane 220.

A radius of curvature for transducer 202 may be, for example, approximately 60 mm (millimeters). The inside (lower, as viewed) surface of transducer 202 may focus acoustic energy, beyond the hemispherical plane HP, beyond bottom surface 224A of membrane 220, into subject body 210. The acoustic energy is represented by the arrows converging on an area 212 within (below surface 210A of) the subject body. Area 212 may be located approximately 2 mm (millimeters) below surface 210A of subject body 210, and may be referred to as “the area being treated”, or “treatment area”.

Transducer (transducing element) 202 may produce therapeutic acoustic energy. Transducing element 202 may include, for example, a piezoelectric element that may be used to produce acoustic waves in response to electrical energy stimulation. The shape, size, thickness, composition and spatial location of transducing element 202 may be adjusted so as to produce a requested acoustic energy. Transducing element 202 may have a substantially dome-like structure. Transducing element 202 may have substantially smooth surfaces (external arched surface and the internal concaved surface), and the thickness of the element may vary, for example, in the range of 0.1 mm to 100 mm. For example, the thickness of transducing element 202 may be in the range of 2 mm to 10 mm. Transducing element 202 may be constructed of various components and formulations that may include such materials as metal, ceramics (PZT), and the like. The dome-like shape of transducing element 202 may allow and aid in focusing the acoustic energy produced by the transducing element.

As a result of electrical energy (or power) provided to transducing element 202, transducing element 202 may vibrate and, as a result, produce acoustic waves and hence acoustic energy. The electrical energy may be provided continuously, and a continuous acoustic wave may be produced. The electrical power provided to transducing element 202 may be provided in pulses/nodes and the resulting vibration energy produced by vibration element 200 may be provided in bursts (such as pulses).

Electrical power provided to transducing element 202 may be in the range of, for example, 1-1000 W (Watts), including but not limited to 1-750 W, 1-500 W, 1-300 W, 1-150 W and 1-100 W. Transducing element 202 may vibrate at a resonance frequency in the range of about 1 to 1200 kHz (kilo-Hertz), including but not limited to about 1 to 1000 kHz, about 1 to 800 kHz, about 1 to 600 kHz, about 1 to 400 kHz, about 1 to 250 kHz, about 1 to 200 kHz, about 1 to 150 kHz, about 1 to 100 kHz and about 1 to 50 kHz.

A focal diameter of transducing element 202, which is the diameter of the region in which the acoustic energy may be focused may be in the range of, for example, about 0.5 to 20 mm (millimeters), including but not limited to about 0.5 to 15 mm, about 0.5 to 12 mm, 0.5 to 10 mm, about 0.5 to 9 mm, about 0.5 to 8 mm, about 0.5 to 7 mm, about 0.5 to 5 mm, about 0.5 to 3 mm, about 0.5 to 2 mm.

A treatment area of the acoustic energy produced by transducing element 202 may be in the range, of for example, the dimensions of the wavelength. For example, the treatment area may have a shape, or volume, such as an oval of approximately one wavelength (short diameter) and two-three wavelengths (long diameter).

A focal distance of the focused acoustic energy produced by transducing element 202 may be measured relative to the working surface, which is the surface to which the energy may be transduced (for example, a subject skin). Thus, the focal distance from the working surface may be in the range of, for example, 1 to 30 mm, including but not limited to 1 to 25 mm, 1 to 20 mm, 1 to 15 mm, 1 to 10 mm, 1 to 5 mm, 1 to 2.5 mm.

An acoustic efficiency of transducing element 202 may be in the range of, for example, about 1 to 150 mg/V (milligram per volt), including but not limited to about 10 to 100 mg/V, about 15 to 75 mg/V, about 20 to 60 mg/V, about 25 to 55 mg/V, about 29 to 50 mg/V.

A peak pressure of transducing element 202, as may be measured at 1 W of electric power per burst (pulse) may be, for example, in the range of 1 to 800 kPa (kilo-Pascal). The peak pressure of transducing element 202, as may be measured at 1 W of electric power per burst (pulse) may be, for example, in the range of 1 to 700 kPa. The peak pressure of transducing element 202, as may be measured at 1 W of electric power per burst (pulse) may be, for example, in the range of 1 to 600 kPa. The peak pressure transducing element 202, as may be measured at 1 W of electric power per burst (pulse) may be, for example, in the range of 100 to 800 kPa. The peak pressure transducing element 202, as may be measured at 1 W of electric power per burst (pulse) may be, for example, in the range of 200 to 700 kPa. The peak pressure of transducing element 202, as may be measured at 1 W of electric power per burst (pulse) may be, for example, in the range of 300 to 600 kPa.

Membrane 220 may transfer the acoustic energy generated by transducing element 202 to subject body 210. Membrane 220 may be comprised of various materials, such as, for example, rubber, plastic, silicon, polyurethane, and the like. Membrane 220 may be comprised of a biocompatible material. For example, membrane 220 may be comprised of a mixture of two polymers or a bi-component polymer. For example, membrane 220 may be comprised of a mixture of soft polyurethane composition TGS 3740 and JG 5803 (purchased from Baule, France). The composition of membrane 220 may be correlated to the acoustic energy that may be transferred through membrane 220. Membrane 220 may have acoustic properties, such as acoustic impedance similar to that of, for example, soft mammalian tissues to which the membrane may transfer the acoustic energy. Membrane 220 may be comprised as one continuous body, or may be comprised of various parts that may be joined together.

Membrane 220 may be uniformly mixed, such that energy transfer through the membrane is uniform and not deviated and/or absorbed by other objects in the membrane composition, such as, for example, air bubbles. For example, membrane 220 may be prepared in a mold. The at-least partially liquid/non-hardened composition of the membrane may be poured into a mold which has a desired shape. Upon polymerization/hardening of membrane 220 (for example, by a chemical reaction, heating, and the like), shaped membrane 220 may be released from the mold and ready to be assembled into the transducer. In addition, a mold release substance may be used, that may aid in releasing the shaped membrane from the mold. The mold release composition may include, for example, a release linear that may be comprised of various non-stick substances, such as silicon.

As best viewed in FIG. 2B, membrane 220 may have a substantially round circular (hemispherical) shape, and may comprise a top (upper) portion 222 and a bottom (lower) portion 224. Top portion 222 is generally that portion of membrane 220 which is above a plane indicated by dashed line 226, and bottom portion 224 is generally that portion of membrane 220 which is below dashed line 226. Dashed line 226 simply represents an arbitrary boundary between the upper and lower portions of the membrane, which would be a plane, and may or may not be the equatorial plane HP of hemispherical membrane 220.

Top portion 222 of membrane 220 is generally hemispherical, and may have an arched, dome-like (substantially hemispherical) shape, and may be referred to as the “dome” of membrane 220. Top portion 222 of membrane 220 has an external surface 222A. A lower region of dome 222 may have a diameter that is substantially equal to the diameter of bottom portion 224 of membrane 220. Moving upwards, the diameter of dome 222 may become increasingly smaller, such as in a sinusoidal function, such that the arched dome-like structure is obtained. The dome-like structure of the top portion of membrane 220 may fit snugly into the concaved area formed by inner surface 202A of transducing element 202. Acoustic transducer 222 may optionally include a hole 223 which may be used for placement of the transducer.

Bottom portion 224 of membrane 220 is generally cylindrical, having a radius and a thickness (height). Bottom portion 224 may have a filled-circular shape with a substantially round circumference. An external surface 224A of bottom portion 224 may be a substantially smooth, substantially planar surface. In use, external surface 224A of bottom portion 224 comes into contact (either directly or indirectly) or in close proximity with surface 110A of subject body 110.

Dashed line 227 represents a plane of bottom surface 224a of bottom portion 224, which is the bottom surface of overall membrane 220, and which may be the hemispherical plane HP. As best viewed in FIG. 2A, treatment area 212, which is within subject body 210, is below the lower extremity (in this example, flat bottom surface 224A) of the membrane, beyond plane 227, such as 2 mm below surface 210A of subject body 210.

A rim, or lip 228 may be disposed about an outer circumference, or periphery of membrane 220, such as immediately above bottom region 224, such as substantially at boundary 226 between upper and lower portions 222 and 224 of membrane 220—in other words, approximately at the top of bottom portion 224.

Rim 228 extends radially outward from the main body of membrane 220, and may thus have a larger diameter than bottom region 222 and thus may extend sideways (radially outwards) as compared to bottom region 222 of membrane 220. Rim 228 may have a substantially round circular ring-like shape. Rim 228 may have two faces: a bottom face 228A that faces subject body 110 surface and a top face 228B that is oriented towards transducing element 202.

A plurality (such as twelve) of pin holes 229 may be disposed (such as evenly spaced) about the circumference of rim 228, in close proximity to the outer circumference of rim 228, and may be used for the securing of membrane 220 to its location in housing 206, for example by the use of screws, pins and the like.

As shown in FIG. 2A, housing (or casing) 206 may be generally in the form of an “inverted cup”. More particularly, the housing may have a generally cylindrical body portion 232 having a top end and a bottom end. Body portion 232 may be closed off at its top end by a generally circular planar surface 234. An annular ridge, or flange 236 may extend radially outward from the bottom end of body portion 232.

Housing 206 is sized to accept (receive) transducer 202, which is disposed therein. Various mechanical details of mounting transducer 202 in housing 206 and connecting transducer 202 to an external power source, and the like, are omitted, for illustrative clarity.

As shown in FIG. 2A, membrane 220 is at least partially disposed within housing 206. Lip 228 of membrane 220 may be in contact with flange 236 extending from the bottom end of housing 206. A separate, annular flange 238 may be provided to secure lip 228 of membrane 220 to flange 236, with fasteners such as screws 239 extending through the corresponding holes 229 in lip 228 of membrane 220.

An intermediate substance and/or material, referred to herein as interposer, may be used, so as to provide an intermediary contact surface between the membrane and the object that may receive the transducing energy, such as a subject body. The interposer may be used to increase efficiency of delivery and/or transmittance of the transducing energy to the subject body. The interposer may include any substance and/or material that may possess such qualities that allow it to be used for the appropriate transmittal of, for example, vibration energy from the transducer to a subject body. The interposer should preferably have such qualities as impendence at a range that corresponds to the vibration energy transduced by the transducer and the appropriate range to be received by the target, such as a subject body. Such substances and materials may include, for example, gel, oil, lubricant, ointment, lotion, water, thin rubber layer and the like, and may be, for example, water based, oil based and the like. For example, the interposer may include ultrasound gel (made by, for example, Medi-Pharm, UK). For example, the interposer used may include castor oil. For example, the interposer used may include paraffin oil. Application of the interposer may be performed by for example, spreading, spraying, laying, pouring or any other appropriate method of application. The interposer may be applied onto the transducer, onto a subject body, or both.

The interposer may include castor oil and it may interact and/or come in contact with, for example, the membrane of the transducer. The use of castor oil as interposer is preferred because of the acoustic impedance that castor oil possesses. The acoustic impedance of castor oil is approximately the same as the acoustic impedance of the polyurethane membrane of the transducer. The similarity in impedance of the castor oil and the polyurethane membrane, maximal vibration energy, such as acoustic energy may be transferred from the transducer to target, such as a subject body.

Vacuum Feature

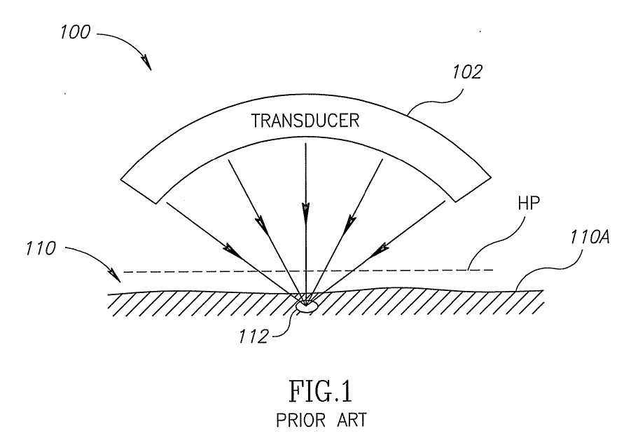

FIG. 1, described hereinabove, is illustrative of an ultrasonic transducer providing focused energy for performing non-invasive, controlled, ultrasound treatment of adipose tissue on a subject body, according to the prior art (such as U.S. Pat. No. 6,607,498).

FIGS. 2A-2B, described hereinabove, are illustrative of an ultrasonic transducer apparatus providing focused energy for performing non-invasive, controlled, ultrasound treatment of adipose tissue on a subject body, generally according to applicant's co-pending patent application. In these figures, some specifics of the transducer, the housing, and the membrane construction are provided.

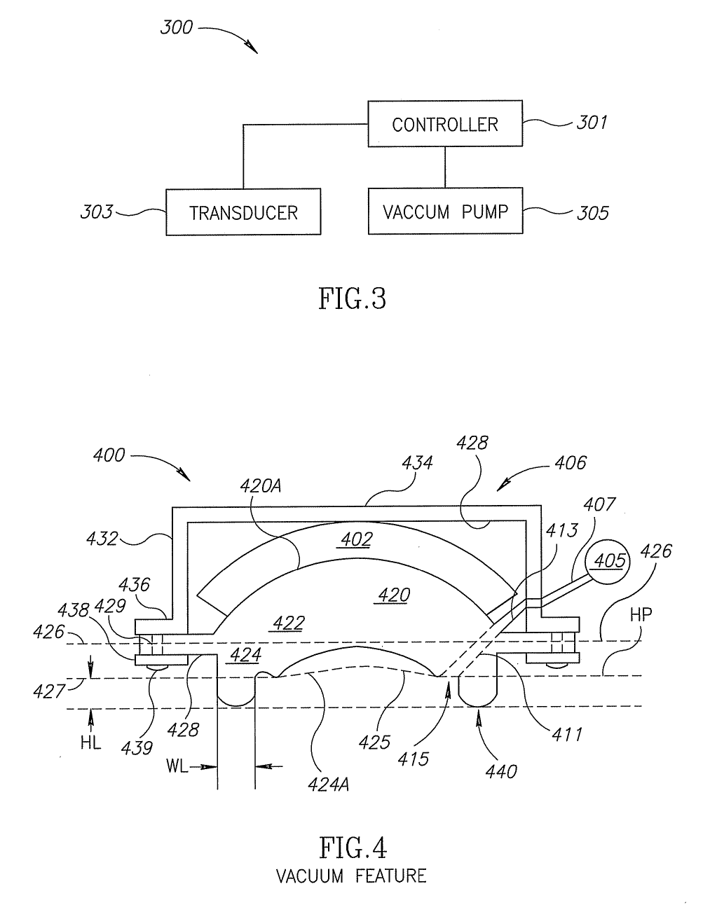

FIG. 3 illustrates an overall system 300 for performing controlled ultrasound treatment of adipose tissue on a subject body.

System 300 comprises a controller 301, such a computer workstation suitably programmed to control and monitor the operation of other components of the system. For example, controller 301 controls the operation of an ultrasonic transducer apparatus 303, such as the transducer apparatus 400 described hereinbelow, such as by providing electrical energy to control the operation of transducer 402 described hereinbelow.

According to an embodiment of the invention, an ultrasonic transducer apparatus is provided with a vacuum feature (suction capability).

Controller 301 may control the operation of a vacuum pump 305, which is part of the overall system, as described in greater detail hereinbelow.

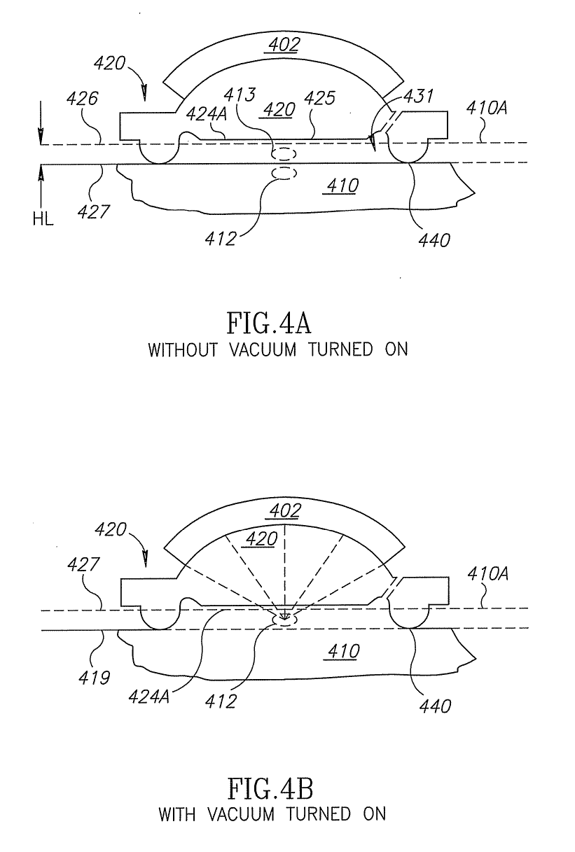

FIG. 4 illustrates a “vibration component” 400 (compare 200) of an overall acoustic transducer apparatus. Main components of vibration component 400 include an acoustic transducer 402 (compare 202), a membrane 420 (compare 220) and a housing (or casing) 406 (compare 206).

A “connection component” (not shown) may be provided to connect transducer 402 to additional devices such as a generator unit that may provide transducer 402 with power, energy, fluids, software instruction, control and feedback, and the like. The vibration component may be used to produce, concentrate and output vibration energy and may come in close proximity and/or contact with a subject body.

Transducer 402 may be substantially identical, in both structure and operation, to transducer 202 described hereinabove. Membrane 420 may be similar to membrane 220, described hereinabove, sharing some features with membrane 220, and having some differences.

A bottom external surface of vibration component 400, which is a bottom surface of membrane 420, is shown spaced slightly from an external surface 410A of the subject body 410 (compare 210), for illustrative clarity.

External casing (housing) 406 of vibration component 400 may be constructed of one integral structure or may be constructed of several constituents that are interconnected to form the external casing of vibration component 400.

Acoustic transducer (or “transducing element”) 402 may have a concave, such as a hemispherical shape (cross-section). An inside surface 402A of the transducer is concave and, in use, is oriented towards a subject body 410. An equatorial plane, indicated by dashed line labeled “HP” may be associated with the hemispherical shape of transducer 402, although transducer 402 is illustrated as not being a complete hemisphere.

The membrane 420 has a bottom surface 424A. The hemispherical plane “HP” may be substantially coincident with a central portion 425 of bottom surface 424a of membrane 420.

A radius of curvature for transducer 402 may be, for example, approximately 60 mm (millimeters). The inside (lower, as viewed) surface of transducer 402 may focus acoustic energy beyond the hemispherical plane HP, into a subject body 410 (see FIGS. 4A and 4B) to treat an area 412 (see FIGS. 4A and 4B) which may be located approximately 2 mm (millimeters) below surface 410A of subject body 410, and may be referred to as “the area being treated”, or “treatment area”. In some cases, that part of surface 410A of subject body 410 which is directly under membrane 420 may be referred to as the “treatment area”.

As illustrated in FIG. 4A, transducer 402 may focus acoustic energy at a point (small area) 413 which is above surface 410A of subject body 410, and which is within the confines of membrane 420.

Transducer (transducing element) 402 may produce therapeutic acoustic energy. Transducing element 402 may include, for example, a piezoelectric element that may be used to produce acoustic waves in response to electrical energy stimulation. The shape, size, thickness, composition and spatial location of transducing element 402 may be adjusted so as to produce a requested acoustic energy. Transducing element 402 may have a substantially dome-like structure. Transducing element 402 may have substantially smooth surfaces (external arched surface and the internal concaved surface), and the thickness of the element may vary, for example, in the range of 0.1 mm to 100 mm. For example, the thickness of transducing element 402 may be in the range of 2 mm to 10 mm. Transducing element 402 may be constructed of various components and formulations that may include such materials as metal, ceramics (PZT), and the like. The dome-like shape of transducing element 402 may allow and aid in focusing the acoustic energy produced by the transducing element.

As a result of electrical energy provided to transducing element 402, transducing element 402 may vibrate and as a result produce acoustic waves and hence acoustic energy. The electrical energy may be provided continuously, and a continuous acoustic wave may be produced. The electrical power provided to transducing element 402 may be provided in pulses/nodes and the resulting vibration energy produced by vibration element 400 may be provided in bursts.

Electrical power provided to transducing element 402 may be in the range of, for example, 1-1000 W (Watts), including but not limited to 1-750 W, 1-500 W, 1-300 W, 1-150 W and 1-100 W. Transducing element 402 may vibrate at a resonance frequency in the range of about 1 to 1200 kHz (kilo-Hertz), including but not limited to about 1 to 1000 kHz, about 1 to 800 kHz, about 1 to 600 kHz, about 1 to 400 kHz, about 1 to 250 kHz, about 1 to 200 kHz, about 1 to 150 kHz, about 1 to 100 kHz and about 1 to 50 kHz.

A focal diameter of transducing element 402, which is the diameter of the region in which the acoustic energy may be focused to may be in the range of, for example, about 0.5 to 20 mm (millimeters), including but not limited to about 0.5 to 15 mm, about 0.5 to 12 mm, 0.5 to 10 mm, about 0.5 to 9 mm, about 0.5 to 8 mm, about 0.5 to 7 mm, about 0.5 to 5 mm, about 0.5 to 3 mm, about 0.5 to 2 mm.

A treatment area of the acoustic energy produced by transducing element 402 may be in the range, of for example, the dimensions of the wavelength. For example, the treatment area may have a shape, or volume, such as an oval of approximately one wavelength (short diameter) and two-three wavelengths (long diameter).

A focal distance of the focused acoustic energy produced by transducing element 402 may be measured relatively to the working surface, which is the surface to which the energy may be transduced (for example, a subject skin). Thus, the focal distance from the working surface may be in the range of, for example, 1 to 30 mm, including but not limited to 1 to 25 mm, 1 to 20 mm, 1 to 15 mm, 1 to 10 mm, 1 to 5 mm, 1 to 2.5 mm.

An acoustic efficiency of transducing element 402 may be in the range of, for example, about 1 to 150 mg/V (milligram per volt), including but not limited to about 10 to 100 mg/V, about 15 to 75 mg/V, about 20 to 60 mg/V, about 25 to 55 mg/V, about 29 to 50 mg/V.

A peak pressure of transducing element 402, as may be measured at 1 W of electric power per burst (pulse) may be, for example, in the range of 1 to 800 kPa (kilo-Pascal). The peak pressure of transducing element 202, as may be measured at 1 W of electric power per burst (pulse) may be, for example, in the range of 1 to 700 kPa. The peak pressure of transducing element 402, as may be measured at 1W of electric power per burst (pulse) may be, for example, in the range of 1 to 600 kPa. The peak pressure of transducing element 402, as may be measured at 1 W of electric power per burst (pulse) may be, for example, in the range of 100 to 800 kPa. The peal pressure of transducing element 402, as may be measured at 1 W of electric power per burst (pulse) may be, for example, in the range of 200 to 700 kPa. The peak pressure of transducing element 402, as may be measured at 1 W of electric power per burst (pulse) may be, for example, in the range of 300 to 600 kPa.

Membrane 420 may be used to transfer the focused acoustic energy generated by transducing element 402 to subject body 410. Membrane 420 may be comprised of various materials, such as, for example, rubber, plastic, silicon, polyurethane, and the like. Membrane 420 may be comprised of a biocompatible material. For example, membrane 420 may be comprised of a mixture of two polymers or a bi-component polymer. For example, membrane 420 may be comprised of a mixture of soft polyurethane composition TGS 3740 and JG 5803 (purchased from Baule, France). The composition of membrane 420 may be correlated to the acoustic energy that may be transferred through membrane 420. Membrane 420 may have acoustic properties, such as acoustic impedance similar to that of, for example, soft mammalian tissues to which the membrane may transfer the acoustic energy. Membrane 420 may be comprised as one continuous body, or may be comprised of various parts that may be joined together.

Membrane 420 may be uniformly mixed, such that energy transfer through the membrane is uniform and not deviated and/or absorbed by other objects in the membrane composition, such as, for example, air bubbles. For example, membrane 420 may be prepared in a mold. The at-least partially liquid/non-hardened composition of the membrane may be poured into a mold which has a desired shape. Upon polymerization/hardening of membrane 420 (for example, by a chemical reaction, heating, and the like), shaped membrane 420 may be released from the mold and be ready to be assembled into the transducer. In addition, a mold release substance may be used, that may aid in releasing the shaped membrane from the mold. The mold release composition may include, for example, a release linear that may be comprised of various non-stick substances, such as silicon.

Membrane 420 may have a substantially round circular (hemispherical) shape, and may comprise a top (upper) portion 422 and a bottom (lower) portion 424. Top portion 422 is generally that portion of membrane 420 which is above a plane indicated by dashed line 426, and bottom portion 424 is generally that portion of membrane 420 which is below dashed line 426. Dashed line 426 simply represents an arbitrary boundary between the upper and lower portions of the membrane, which would be a plane, and may or may not be the equatorial plane HP of hemispherical membrane 420.

Top portion 422 of membrane 420 is generally hemispherical, and may have an arched, dome-like (substantially hemispherical) shape, and may be referred to as the “dome” of membrane 420. A lower region of dome 422 may have a diameter that is substantially equal to the diameter of bottom portion 424 of membrane 420. Moving upwards, the diameter of dome 422 may become increasingly smaller, such as in a sinusoidal function, such that the arched dome-like structure is obtained. The dome-like structure of the top portion of membrane 420 may fit snugly into the concaved area formed by inner surface 402A of transducing element 402.

Whereas external surface 424A of bottom portion 424 of membrane 420 is substantially entirely flat, the external (bottom) surface 424A of membrane 420 is only partially flat, in a central region 425 thereof. Central region 425 may be a substantially smooth, substantially planar surface.

Annular lip 440, described in greater detail hereinbelow, extends downward (protrudes) from a peripheral region of bottom surface 424A of the membrane. A dashed line 427 (compare 227) indicates the level of a plane defined by the bottom end surface of annular lip 440, which may be the lowest part of apparatus 400, hence the first to establish contact with surface 410A of the subject body.

A rim, or lip 428 may be disposed about an outer circumference, or periphery of membrane 420, such as immediately above bottom region 424, such as substantially at boundary 426 between the upper and lower portions 422 and 424 of membrane 420—in other words, approximately at the top of bottom portion 424.

Rim 428 extends radially outward from the main body of membrane 420, and may thus have a larger diameter than bottom region 422 and thus may extend sideways (radially outwards) as compared to bottom region 422 of membrane 422. Rim 428 may have a substantially round circular ring-like shape. Rim 428 may have two faces: a bottom face that faces the subject body 110 surface and a top face that is oriented towards the transducing element 402.

A plurality (such as twelve) of pin holes 429 may be disposed (such as evenly spaced) about the circumference of rim 428, in close proximity to the outer circumference of 428, and may be used for the securing of membrane 420 to its location in housing 406, for example by the use of screws, pins and the like.

Housing (or casing) 406 may be generally in the form of an “inverted cup”. More particularly, housing 406 may have a generally cylindrical body portion 432 having a top end and a bottom end. Body portion 432 may be closed off at its top end by a generally circular planar surface 434. An annular ridge, or flange 436 may extend radially outward from the bottom end of the body portion 432.

Housing 406 is sized to accept (receive) transducer 402, which is disposed therein. Various mechanical details of mounting transducer 402 in housing 406 and connecting transducer 402 to an external power source, and the like, are omitted, for illustrative clarity.

As shown in FIG. 4, membrane 420 is at least partially disposed within housing 406. Lip 428 of membrane 420 may be in contact with flange 436 extending from the bottom end of housing 406. A separate, annular flange 438 may be provided to secure lip 428 of membrane 420 to flange 436, with fasteners such as screws 439 extending through corresponding holes 429 in lip 428 of membrane 420.

Annular lip 440 protrudes from an outer, peripheral, circumferential portion of bottom surface 424A of membrane 420. Dashed line 427 indicates the level of a plane defined by annular lip 440. Annular lip 440 has a height dimension “HL”, which is shown as the offset between the two dashed lines 426 and 427. Annular lip 440 has a width dimension, labeled “WL”. Annular lip 440 may be formed integrally with at least bottom portion 424 of membrane 420.

Dashed line 427 also indicates a nominal level of surface 410A of subject body 410. (For purposes of this discussion, surface 410A of subject body 410 is assumed to be substantially flat in an area covered by membrane 420. For a membrane 420 having a circumference of approximately 70 -200 mm (such as, 90-110 mm), this is a reasonable assumption when surface 410A of the subject body 410 is the skin of a human patient, such as in an abdominal area.

The height dimension “HL” of the lip 440 may be 1-20 mm (such as, 5-10 mm). The width dimension “WL” of the lip 440 may be 1-20 mm (such as, 5-10 mm).

Annular lip 440, serves to offset the flat, central region 425 of bottom surface 424a of membrane 420 initially away from surface 410A of subject body 410, forming (defining) an enclosed space 431 between bottom surface 424A of membrane 420 and surface 410A of subject body 410. This is best viewed in FIG. 4A.

According to some embodiments of the invention, a vacuum pump 405 (compare 305) is connected by at least one vacuum line 407 membrane 420, and membrane 420 has at least one passageway 411, extending from outer surface 420A to inner surface 424A thereof and, more particularly, to enclosed space 431 between membrane 420 and the surface of the subject body. A tube 413 may be provided within housing 406 to convey vacuum from source line 407, through an exterior surface of the housing (see dashed lines), to passageway 411 in the membrane. A channel (trough, recess) 415 may be provided around the inner edge of annular lip 440, extending (intruding) slightly into central region 425 of bottom surface 424A of membrane 420, to ensure that vacuum can be distributed substantially evenly around enclosed space 431. Annular lip 440 protrudes from bottom surface 424A of membrane 420 around central region 425, and channel 415 intrudes into central region 425 just within annular lip 440. Vacuum pump 405, in cooperation with membrane 420 and the enclosed space defined therein (and passageway 411, and the tube) constitute a “drawing element” (means) for drawing a surface of the subject body into overall vibration component 400, particularly into enclosed space 432 in membrane (acoustic coupling) element 420 of vibration component 400. (The overall vibration component 400 may be referred to herein as “transducer”.)

FIG. 4A illustrates membrane 420 (and transducer 402) placed in contact with subject body 410. An area of surface 410A of subject body 410 which is directly under membrane 420, particularly under central region 425 of membrane 420, may be referred to as a “contact area”. An ultrasonic gel (not shown) may be used to ensure good contact. In this figure, vacuum 405 is not turned on, and enclosed space 431 formed by central region 425 of bottom surface 424A of membrane 420, annular lip 440, and surface 410A of subject body 410 is apparent. Surface 410A of subject body 410 may be substantially flat and, if human skin tissue, is resilient.

As is evident in FIG. 4A, a focal point 413 of the acoustic energy produced by transducer 402 is within the confines of membrane 420, in enclosed space 431 (whether or not space 431 is enclosed, focal point 413 is there), and is indicated by dashed line ellipse 413. If there were no vacuum, and surface 410A of subject body 410 were not deformed, this position of focal point 413, within the apparatus (versus without the apparatus) may not be efficacious, since it would be outside of (above) the surface of treatment area 412.

FIG. 4B illustrates membrane 420 (and transducer 402) placed in contact with subject body 410. In this figure, vacuum 405 is turned on. In use, when membrane 420 is placed in contact with subject body 410 and vacuum pump 405 is turned on, a portion of surface 410A of subject body 410 (such as a patients skin tissue) may be drawn (sucked) up into enclosed space 431 between the membrane and the surface of the subject body. (This may be referred to as “grabbing” the tissue being treated.) Along therewith, treatment area 412 (compare 112) which may be located approximately 2 mm (millimeters) below surface 410A of subject body 410, may be drawn towards transducer 420, into enclosed space 431 between membrane 420 and surface 410A of subject body 410, and above the level of the rest of surface 410A of subject body 410. With the vacuum turned on, treatment area 412 may be substantially coincident with focal point 413 (see FIG. 4A), within enclosed space 431.

This feature, that surface 410A of subject body 410 may be deformed so that treatment area (target tissue) 412 is moved (or sucked up) to be (fixed) within the confines of membrane 420, may be useful for avoiding damage to tissue or organs adjacent (particularly deeper within the subject body) than treatment area 412. It is, however, within the scope of the invention that although surface 410A of subject body 410 may be deformed by supplying vacuum, treatment area (target tissue) 412 may still be outside (not within the confines) of the membrane, such as below plane 419.

This deformation of the target tissue may result in pre-stressing of the target tissue for intensification of natural body reactions (blood flow, lymphatic drainage, and so forth).

In addition to possibly drawing surface 410A of subject body 410 up into the confines of membrane 420, by supplying vacuum, and by having good contact between annular lip 440 and surface 410A of subject body 410, external atmospheric pressure will tend to hold membrane 420 securely against surface 410A of subject body 410.

FIG. 4C illustrates, according to alternate embodiments, a “vibration component” 400C of an overall acoustic transducer apparatus. “Vibration component” 400C is similar to “vibration component” 400 illustrated in FIG. 4, with some differences. Main components of vibration component 400C include an acoustic transducer 402C, having a through hole 423, which may be used, for example, to properly place the transducer. Main components of vibration component 400C include a membrane 420C which may have a substantially round circular (hemispherical) shape, and may comprise a top (upper) portion 422C and a bottom (lower) portion 424C, bottom surface 424C being curved at a central portion 425C relative to the hemispherical plane “HP”. Top portion 422C is generally that portion of membrane 420C which is above a plane indicated by dashed line 426, and bottom portion 424C is generally that portion of membrane 420C which is below the dashed line 426. Top portion 422C may further include a channel 423C adapted to transfer fluids (such as acoustic, contact fluid, lubricant, drug containing fluid or any other type of fluid).

In FIG. 4, membrane 420 is shown with an annular lip 440 having a simple, inverted, substantially hemispherical profile (cross-section). Various other profiles for the annular lip are possible, a few of which are illustrated in the following figures.

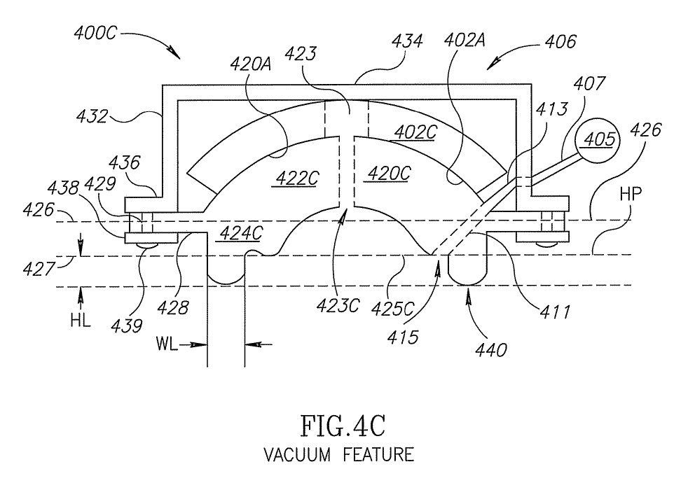

FIG. 5A illustrates a bottom portion 525A of a membrane 520A, (such as membrane 420, in FIG. 4), illustrating an annular lip 540A having a simple, inverted, substantially hemispherical profile (cross-section). Membrane 520A has at least one passageway 531A, extending from bottom portion 525A to inner surface 524A thereof allowing vacuum suction.

FIG. 5B illustrates a bottom portion 525B of a membrane 520B, (such as membrane 420, in FIG. 4) illustrating an annular lip 540B having a substantially rectangular profile (cross-section). Membrane 520B has at least one passageway 530B, extending from bottom portion 525B to inner surface 524B thereof allowing vacuum suction.

FIG. 5C illustrates a bottom portion 525C of a membrane 520C, (such as membrane 420, in FIG. 4) illustrating an annular lip 540C having an inverted, substantially U-shaped profile (cross-section). Membrane 520C has at least two passageways 530C and 531C, extending from bottom portion 525C to inner surface 524C thereof allowing vacuum suction.

The vacuum (suction) feature allows for the transducer to “grab” the fat tissue (through the skin) with good vacuum acoustic contact between the tissue and the transducer.

Since the acoustic contact between the tissue and the transducer is increased, it is possible to use lubricants with lower viscosity than castor oil, for example baby oil. This may reduce pain experienced by the patient during treatment, and also may make it easier for the doctor to slide the transducer. In addition, as explained above castor oil reacts with the transducer's membrane. The vacuum feature may also facilitate treating hairy patients.

According to some embodiments, the vacuum can also be used to massage the tissue, which may assist in the reduction of fat (for example, LPG massage).

According to some embodiments, the vacuum can also operate in a pulse mode, for example to increase the massage effect. For example, turning the vacuum “on” every 1-20 seconds (such as, 3-10 seconds) for 1-20 seconds (such as, 3-10 seconds), and allowing the vacuum to subside, between these “on” times. Regarding allowing the vacuum to subside, a vent (not shown) in the vacuum pump or in the transducer apparatus may be opened to allow the pressure within enclosed space 431 to return to atmospheric pressure.

In addition, the pulse mode may be used as follows: the vacuum is on when the transducer is in contact with the body tissue and off when it is moved to another spot on the tissue.

Moreover, the suction of the tissue into the transducer may allow for better focusing of the energy to the fat tissue, as opposed to focusing the energy (or even emitting part of the energy) to internal organs, which is not desired.

According to some embodiments, the curved shape of the transducer's membrane (the part that is in contact with the body) may allow for suction of more fat tissue into the transducer and further improve the focusing of the energy to the fat tissue, as opposed to emitting energy to internal organs.

According to some embodiments, vibration component 400 may further include a fluid injection unit, described in greater detail hereinbelow.

Fluid Injection

FIG. 1, described hereinabove, is illustrative of an ultrasonic transducer providing focused energy for performing non-invasive, controlled, ultrasound treatment of adipose tissue on a subject body, according to the prior art (such as U.S. Pat. No. 6,607,498).

FIGS. 2A-2B, described hereinabove, are illustrative of an ultrasonic transducer apparatus providing focused energy for performing non-invasive, controlled, ultrasound treatment of adipose tissue on a subject body, generally according to applicant's co-pending patent application. In these figures, some specifics of the transducer, the housing, and the membrane construction are provided.

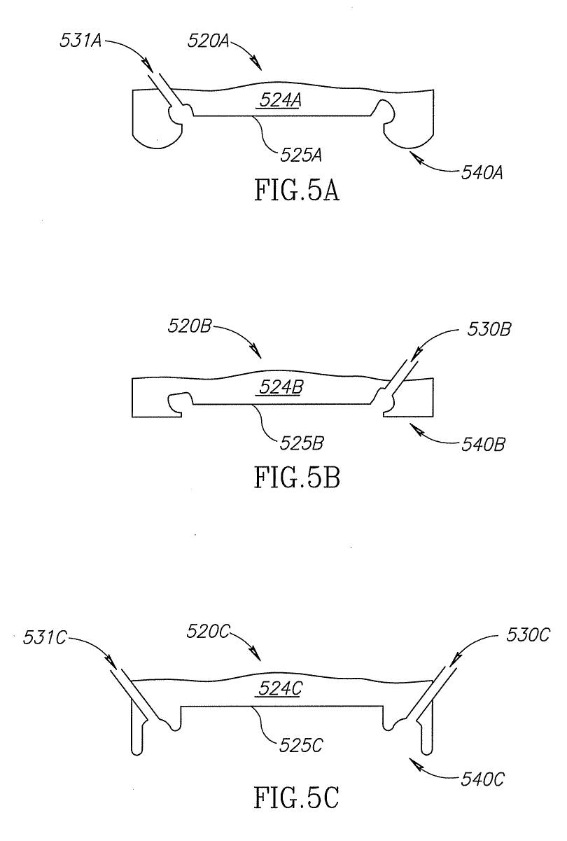

FIG. 6 illustrates an overall system 600 (compare 300) for performing controlled ultrasound treatment of adipose tissue on a subject body.

System 600 comprises a controller 601 (compare 301), such a computer workstation suitably programmed, to control and monitor the operation of other components of the system. For example, controller 601 controls the operation of an ultrasonic transducer apparatus 603 (compare 303), such as transducer apparatus 700 described hereinbelow, such as by providing electrical energy to control the operation of transducer 702, as described hereinbelow.

According to an embodiment of the invention, an ultrasonic transducer apparatus is provided with fluid injection (delivery and flow) capability.

Controller 601 may control the operation of one or more fluid pumps 608, which is part of the overall system, as described in greater detail hereinbelow.

Generally, transducer 702 includes a fluid injection unit that is adapted to inject the lubrication fluid (optionally continuously) to the treatment area, particularly to an area of the surface of the subject body which is immediately below the central region of the membrane. Since the lubrication fluid is circulating (flowing in and out of the contact point between the membrane and the treatment area), contact acoustic fluids (or lubricants) which are less viscous than castor oil may be used, particularly if vacuum is added to suck the fluid and prevent it from leaking. It is thus possible to use lubricants with lower viscosity than castor oil, for example baby oil or even water. This may reduce the pain during treatment and also may make it easier for the doctor to slide the transducer across the surface of the subject body. In addition, the use of a fluid other than castor oil will avoid a problem of castor oil reacting with the membrane.

Additionally, the circulation of the fluid also solves the hygiene problem, since every patient may be treated with new, unused lubricant. Moreover, the lubricant may include additional substances, such as drugs, fat reducing agents, pain relief agents, analgesics, hair removal agents and others. This may be particularly beneficial since the ultrasonic energy may facilitate the penetration of these substances to deeper layers of the tissue—for example, for uprooting hair. To further enhance the penetration effect different energy frequencies may be used.

The transducer may also have a vacuum feature, as described hereinabove, that allows “grabbing” of the fat tissue (through the skin) with good vacuum acoustic contact between the tissue and the transducer, as described hereinabove.

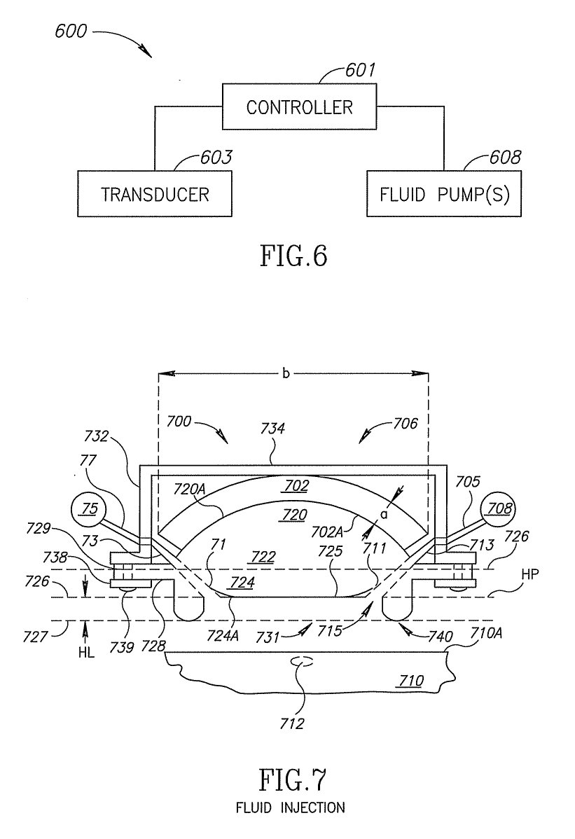

FIG. 7 illustrates a “vibration component” 700 (compare 400) of an overall acoustic transducer apparatus. Main components of vibration component 700 include an acoustic transducer 702 (compare 402), a membrane 720 (compare 420) and a housing (or casing) 706 (compare 406).

A “connection component” (not shown) may be provided to connect transducer 702 to additional devices such as a generator unit that may provide transducer 702 with power, energy, fluids, software instruction, control and feedback, and the like. The vibration component may be used to produce, concentrate and output vibration energy and may come in close proximity and/or contact with a subject body.

Transducer 702 may be substantially identical, in both structure and operation, to transducer 402, described hereinabove. Membrane 720 may be similar to membrane 420, described hereinabove, sharing some features with membrane 420, and having some differences.

A bottom external surface of vibration component 700, which is a bottom surface of membrane 720, is shown spaced slightly from an external surface 710A of subject body 710 (compare 210), for illustrative clarity.

External casing (housing) 706 of vibration component 700 may be constructed of one integral structure or may be constructed of several constituents that are interconnected to form the external casing of vibration component 700.

Acoustic transducer (or “transducing element”) 702 may have a concave, such as a hemispherical shape (cross-section). An inside surface 702A of the transducer is concave and, in use, is oriented towards a subject body 710. An equatorial plane, indicated by dashed line labeled “HP” may be associated with the hemispherical shape of transducer 702, although transducer 702 is illustrated as not being a complete hemisphere.

Membrane 720 has a bottom surface 724a. The hemispherical plane “HP” may be substantially coincident with a central portion 725 of bottom surface 724A of membrane 720.

A radius of curvature for transducer 702 may be, for example, approximately 60 mm (millimeters). The inside (lower, as viewed) surface of transducer 702 may focus acoustic energy beyond the hemispherical plane HP, into subject body 710 to treat area 712 which may be located approximately 2 mm (millimeters) below surface 710A of subject body 710, and may be referred to as “the area being treated”, or “treatment area”. In some cases, that part of surface 710A of subject body 710 which is directly under membrane 720 may be referred to as the “treatment area”.

As discussed hereinabove, transducer 702 may focus acoustic energy at a point (small area) which is above surface 710A of subject body 710, and which is within the confines of membrane 720.

Transducer (transducing element) 702 may produce therapeutic acoustic energy. Transducing element 702 may include, for example, a piezoelectric element that may be used to produce acoustic waves in response to electrical energy stimulation. The shape, size, thickness, composition and spatial location of transducing element 702 may be adjusted so as to produce a requested acoustic energy. Transducing element 702 may have a substantially dome-like structure. Transducing element 702 may have substantially smooth surfaces (external arched surface and the internal concaved surface), and the thickness of the element may vary, for example, in the range of 0.1 mm to 100 mm. For example, the thickness of transducing element 702 may be in the range of 2 mm to 10 mm. The transducing element may be constructed of various components and formulations that may include such materials as metal, ceramics (PZT), and the like. The dome-like shape of transducing element 702 may allow and aid in focusing the acoustic energy produced by the transducing element.

As a result of electrical energy provided to transducing element 702, transducing element 702 may vibrate and as a result produce acoustic waves and hence acoustic energy. The electrical energy may be provided continuously, and a continuous acoustic wave may be produced. The electrical power provided to transducing element 702 may be provided in pulses/nodes and the resulting vibration energy produced by vibration element 700 may be provided in bursts.

Electrical power provided to transducing element 702 may be in the range of, for example, 1-1000 W (Watts), including but not limited to 1-750 W, 1-500 W, 1-300 W, 1-150 W and 1-100 W. Transducing element 702 may vibrate at a resonance frequency in the range of about 1 to 1200 kHz (kilo-Hertz), including but not limited to about 1 to 1000 kHz, about 1 to 800 kHz, about 1 to 600 kHz, about 1 to 400 kHz, about 1 to 250 kHz, about 1 to 200 kHz, about 1 to 150 kHz, about 1 to 100 kHz and about 1 to 50 kHz.

A focal diameter of transducing element 702, which is the diameter of the region in which the acoustic energy may be focused may be in the range of, for example, about 0.5 to 20 mm (millimeters), including but not limited to about 0.5 to 15 mm, about 0.5 to 12 mm, 0.5 to 10 mm, about 0.5 to 9 mm, about 0.5 to 8 mm, about 0.5 to 7 mm, about 0.5 to 5 mm, about 0.5 to 3 mm, about 0.5 to 2 mm.

A treatment area of the acoustic energy produced by transducing element 702 may be in the range, of for example, the dimensions of the wavelength. For example, the treatment area may have a shape, or volume, such as an oval of approximately one wavelength (short diameter) and two-three wavelengths (long diameter).

A focal distance of the focused acoustic energy produced by transducing element 702 may be measured relative to the working surface, which is the surface to which the energy may be transduced (for example, a subject skin). Thus, the focal distance from the working surface may be in the range of, for example, 1 to 30 mm, including but not limited to 1 to 25 mm, 1 to 20 mm, 1 to 15 mm, 1 to 10 mm, 1 to 5 mm, 1 to 2.5 mm.

An acoustic efficiency of transducing element 702 may be in the range of, for example, about 1 to 150 mg/V (milligram per volt), including but not limited to about 10 to 100 mg/V, about 15 to 75 mg/V, about 20 to 60 mg/V, about 25 to 55 mg/V, about 29 to 50 mg/V.

A peak pressure of transducing element 702, as may be measured at 1 W of electric power per burst (pulse) may be, for example, in the range of 1 to 800 kPa (kilo-Pascal). The peak pressure of transducing element 702, as may be measured at 1 W of electric power per burst (pulse) may be, for example, in the range of 1 to 700 kPa. The peak pressure of transducing element 702, as may be measured at 1 W of electric power per burst (pulse) may be, for example, in the range of 1 to 600 kPa. The peak pressure of transducing element 702, as may be measured at 1 W of electric power per burst (pulse) may be, for example, in the range of 100 to 800 kPa. The peak pressure of transducing element 702, as may be measured at 1 W of electric power per burst (pulse) may be, for example, in the range of 200 to 700 kPa. The peak pressure of transducing element 702, as may be measured at 1 W of electric power per burst (pulse) may be, for example, in the range of 300 to 600 kPa.

Membrane 720 may be used to transfer the focused acoustic energy generated by transducing element 702 to subject body 710. Membrane 720 may be comprised of various materials, such as, for example, rubber, plastic, silicon, polyurethane, and the like. Membrane 720 may be comprised of a biocompatible material. For example, membrane 720 may be comprised of a mixture of two polymers or a bi-component polymer. For example, membrane 720 may be comprised of a mixture of soft polyurethane composition TGS 3740 and JG 5803 (purchased from Baule, France). The composition of membrane 720 may be correlated to the acoustic energy that may be transferred through membrane 720. Membrane 720 may have acoustic properties, such as acoustic impedance similar to that of, for example, soft mammalian tissues to which the membrane may transfer the acoustic energy. Membrane 720 may be comprised as one continuous body, or may be comprised of various parts that may be joined together.

Membrane 720 may be uniformly mixed, such that energy transfer through the membrane is uniform and not deviated and/or absorbed by other objects in the membrane composition, such as, for example, air bubbles. For example, membrane 720 may be prepared in a mold. The at-least partially liquid/non hardened composition of the membrane may be poured into a mold which has a desired shape. Upon polymerization/hardening of membrane 720 (for example, by a chemical reaction, heating, and the like), shaped membrane 720 may be released from the mold and ready to be assembled into the transducer. In addition, a mold release substance may be used, that may aid in releasing the shaped membrane from the mold. The mold release composition may include, for example, a release linear that may be comprised of various non-stick substances, such as silicon.

Membrane 720 may have a substantially round circular (hemispherical) shape, and may comprise a top (upper) portion 722 and a bottom (lower) portion 724. Top portion 722 is generally that portion of membrane 720 which is above a plane indicated by dashed line 726, and bottom portion 724 is generally that portion of membrane 720 which is below dashed line 726. Dashed line 726 simply represents an arbitrary boundary between the upper and lower portions of the membrane, which would be a plane, and may or may not be the equatorial plane HP of hemispherical membrane 720.

Top portion 722 of membrane 720 is generally hemispherical, and may have an arched, dome-like (substantially hemispherical) shape, and may be referred to as the “dome” of membrane 720.

A lower region of dome 722 may have a diameter that is substantially equal to the diameter of bottom portion 724 of membrane 720. Moving upwards, the diameter of dome 722 may become increasingly smaller, such as in a sinusoidal function, such that the arched dome-like structure is obtained. The dome-like structure of the top portion of membrane 720 may fit snugly into the concaved area formed by inner surface 702A of transducing element 702.

Whereas in xternal surface 724A of bottom portion 724 the membrane 720 is substantially entirely flat, external (bottom) surface 724A of membrane 720 is only partially flat, in a central region 725 thereof. Central region 725 may be a substantially smooth, substantially planar surface.

An annular lip 740, described in greater detail hereinbelow, extends downward (protrudes) from a peripheral region of bottom surface 724A of the membrane. A dashed line 727 (compare 227) indicates the level of a plane defined by the bottom end surface of annular lip 740, which may be the lowest part of apparatus 700, hence the first to establish contact with surface 710A of the subject body.

A rim, or lip 728 may be disposed about an outer circumference, or periphery of membrane 720, such as immediately above bottom region 724, such as substantially at boundary 726 between upper and lower portions 722 and 724 of membrane 720—in other words, approximately at the top of bottom portion 724.

Rim 728 extends radially outward from the main body of membrane 720, and may thus have a larger diameter than bottom region 722 and thus may extend sideways (radially outwards) as compared to bottom region 722 of membrane 722. Rim 728 may have a substantially round circular ring-like shape. Rim 728 may have two faces: a bottom face that faces subject body 110 surface and a top face that is oriented towards transducing element 702.

A plurality (such as twelve) of pin holes 729 may be disposed (such as evenly spaced) about the circumference of rim 728, in close proximity to the outer circumference of rim 728, and may be used for the securing of membrane 720 to its location in housing 706, for example by the use of screws, pins and the like.

Housing (or casing) 706 may be generally in the form of an “inverted cup”. More particularly, housing 706 may have a generally cylindrical body portion 732 having a top end and a bottom end. Body portion 732 may be closed off at its top end by a generally circular planar surface 734. An annular ridge, or flange 736 may extend radially outward from the bottom end of body portion 732.

Housing 706 is sized to accept (receive) transducer 702, which is disposed therein. Various mechanical details of mounting transducer 702 in housing 706 and connecting transducer 702 to an external power source, and the like, are omitted, for illustrative clarity.

As shown in FIG. 7, membrane 720 is at least partially disposed within housing 706. Lip 728 of membrane 720 may be in contact with flange 736 extending from the bottom end of housing 706. A separate, annular flange 738 may be provided to secure lip 728 of membrane 720 to flange 736, with fasteners such as screws 739 extending through the corresponding holes 729 in lip 728 of membrane 720.

Annular lip 740 protrudes from an outer, peripheral portion of bottom surface 724A of membrane 720. Dashed line 727 indicates the level of a plane defined by annular lip 740. Annular lip 740 may be substantially identical to annular lip 440, including the profile variations shown and described with respect to FIGS. 5A-5C. Annular lip 740 has a height dimension “HL”, and has a width dimension, labeled “WL” (see FIG. 4). Annular lip 740 may be formed integrally with at least bottom portion 724 of membrane 720.

The dashed line 727 also indicates a nominal level of the surface 710A of the subject body 710. (For purposes of this discussion, the surface 710A of subject body 710 is assumed to be substantially flat in an area covered by membrane 720.

For example, the dimension “b” may be in the range of 50-150 mm, such as, 75-100 mm, about 95 mm. The dimension “a” may be in the range of 0.1-10 mm, such as, 1-7 mm or about 7 mm (which may corresponds to a frequency of 200 kHz). Annular lip 740, serves to offset the flat, central region 725 of bottom surface 724A of the membrane initially away from surface 710A of subject body 710, forming (defining) an enclosed space 731 (compare 431, FIG. 4A) between membrane 720 and surface 710A of subject body 710.

According to some embodiments of the invention, fluid may be supplied to enclosed space 731 between membrane 720 and surface 710A of subject body 710. An area of surface 710A of subject body 710 which is directly under membrane 720, particularly under central region 725 of membrane 720, may be referred to as a “contact area”.

A fluid supply 75 is connected by a line 77 to membrane 720, and membrane 720 has at least one passageway 71, extending from outer surface 720A to inner surface 724A thereof and, more particularly, to enclosed space 731 between the membrane and surface 710A of subject body 710. A tube 73 may be provided within housing 706 to convey fluid from line 77, through an exterior surface of the housing (see dashed lines), to passageway 71 in the membrane.

A channel (trough, recess) 715 may be provided around the inner edge of annular lip 740, extending (intruding) slightly into central region 725 of bottom surface 724A of membrane 720, to ensure that fluid can be distributed substantially evenly around enclosed space 731. Annular lip 740 protrudes from bottom surface 724A of membrane 720 around central region 725, and channel 715 intrudes into central region 725 just within annular lip 740.

A fluid collector (reservoir) 708 is connected by a line 705 to membrane 720, and membrane 720 has at least one passageway 711, extending from outer surface 720A to inner surface 724A thereof and, more particularly, to enclosed space 731 between the membrane and the surface of the subject body. A tube 713 may be provided within housing 706 to convey fluid from passageway 711 in membrane 720 to line 707.

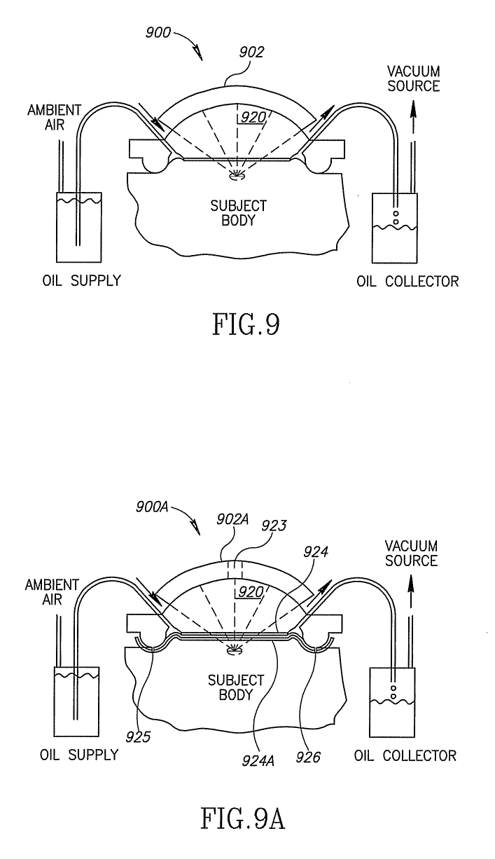

In this manner fluid may be provided, such as continuously, through vibration component 700, to a “contact area” on surface 710A of subject body 710. Fluid supply 75 constitutes at least a portion of an overall “dispenser” (or means) for supplying a fluid to the contact area on the surface of a subject body. The fluid (not shown), which is supplied by the fluid supply, may be contact acoustic fluid and/or drugs. The dispenser may comprise a supply reservoir and a pump. Alternatively, as illustrated in FIG. 9, a vacuum source may be provided to cause fluid from a fluid (such as oil) supply to be supplied to the contact area under the membrane.

FIG. 7A illustrates a “vibration component” 700A of an overall acoustic transducer apparatus, which is similar to “vibration component” 700 of an overall acoustic transducer apparatus, shown in FIG. 7, with some differences. Main components of vibration component 700A include an acoustic transducer 702A, a membrane 720A and a space 723. Membrane 720A may comprise a top (upper) portion 722A and a bottom (lower) portion 724A. Top portion 722A and acoustic transducer 702A define space 723 which may be filled, for example with oil or any other appropriate substance that may have minimal effect on the intensity or characteristics of the signals produced by the transducer, and may be impedance matched to the characteristics of the acoustic energy emitted by the transducer. Membrane 720A may comprise polyethylene, nylon or any other material that may have minimal effect on the intensity or characteristics of the signals produced by the transducer, and may be impedance matched to the characteristics of the acoustic energy emitted by the transducer.

The dimension “c” may be in the range of 1-5 mm, such as 1.2 mm, which may correspond to a frequency of 1 MHz.

According to some embodiments, vibration component 700 may further include a vacuum feature (source), described in greater detail hereinabove. The vacuum feature can perform the functions associated with sucking target tissue up into the transducer, as well as being used to cause fluid flow from the fluid supply.

As used herein, “transducer” may refer to the ultrasonic transducer element or component (402, 702) itself, or to the overall vibration component 400, 700. As used herein (in at least some of the embodiments which may be described herein), concave (curved) inner (inside) surface (402A, 702A) of ultrasonic transducer (420, 720) may constitute an element (or “means”) for producing, providing, directing and/or focusing acoustic energy or waves at a (focal) distance from the curved surface, typically at a target area (treatment area 412, 712) which is within a surface (410A, 710A) of a subject body (410, 710). Generally, transducer (402, 702) emits acoustic energy with an intensity sufficient for cavitational or mechanical lysis of the target tissue. The transducer may be actuated to transmit periodic ultrasonic waves of sufficient intensity ultrasonic waves of sufficient intensity to cause cavitation and lysis of said adipose tissue without damaging adjacent non-adipose tissue.

As used herein, “element” may refer to one or more elements. For example, the dispenser element for supplying a fluid, through the transducer, to a contact area on the surface of a subject body may comprise a fluid supply reservoir and a pump, and may also comprise a vacuum source.

The process for treatment of adipose tissue may be enhanced by applying a vacuum to the transducer to suck the target tissue up into the transducer and by supplying acoustic contact fluid and/or drugs to the treatment area. The vacuum may also be pulsed, to massage (physically stimulate) the treatment area.

Vacuum Feature and Fluid Injection

FIG. 1, described hereinabove, is illustrative of an ultrasonic transducer providing focused energy for performing non-invasive, controlled, ultrasound treatment of adipose tissue on a subject body, according to the prior art (such as U.S. Pat. No. 6,607,498). Alternative, non-prior shapes and parameters have been described previously while others will be apparent to those with skill in the art.

FIGS. 2A-2B, described hereinabove, are illustrative of an ultrasonic transducer apparatus providing focused energy for performing non-invasive, controlled, ultrasound treatment of adipose tissue on a subject body, generally according to applicant's co-pending patent application. In these figures, some specifics of the transducer, the housing, and the membrane construction are provided.

FIG. 3, described hereinabove, illustrates an overall system for performing controlled ultrasound treatment of adipose tissue on a subject body, with a vacuum feature. Refer also to FIGS. 4, 4A, and 4B, and the accompanying descriptions thereof.

FIG. 6, described hereinabove, illustrates an overall system for performing controlled ultrasound treatment of adipose tissue on a subject body, with fluid injection. Refer also to FIG. 7, and the accompanying description thereof.

According to an embodiment of the invention, an ultrasonic transducer apparatus is provided with a vacuum feature (suction capability), and with fluid injection.

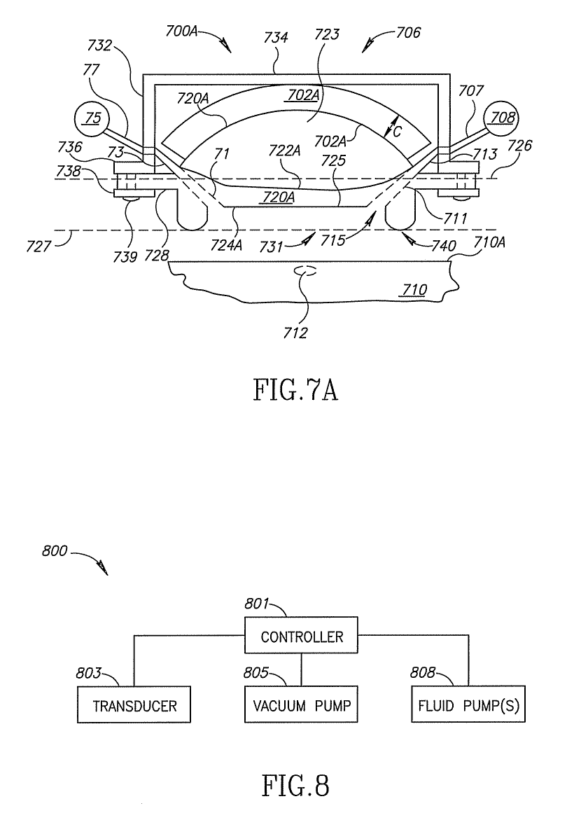

FIG. 8 illustrates an overall system 800 (compare 300, compare 600) for performing controlled ultrasound treatment of adipose tissue on a subject body.

System 800 comprises a controller 801 (compare 301, compare 601), such a computer workstation suitably programmed, to control and monitor the operation of other components of the system. For example, controller 801 controls the operation of an ultrasonic transducer apparatus 803 (compare 303, compare 603), such as transducer apparatus 90.0, described hereinbelow, such as by providing electrical energy to control the operation of transducer 902 described hereinbelow.

Controller 801 may control the operation of a vacuum pump 805 (compare 305), which is part of the overall system, as described in greater detail hereinbelow.

Controller 801 may control the operation of one or more fluid pumps 808 (compare 608), which is part of the overall system, as described in greater detail hereinbelow.

FIG. 9 illustrates a system 900 combining the features of vacuum and fluid injection. A transducer 902 (compare 402, compare 702) is provided. A membrane 920 (compare 420, compare 720), and has an annular lip as described hereinabove (refer to 440, 740) to form an enclosed space (refer to 431, 731) between the membrane and a surface of the subject body.

Generally, referring back to and combining some of the features discussed hereinabove, a method and apparatus 900 for treatment of adipose tissue may comprise a transducer 902 directing acoustic energy at a subject body and a membrane 920 disposed between transducer 902 and the subject body. Membrane 920 may include an annular lip (440, 640) for establishing an enclosed space (431, 731) between the membrane and the surface of the subject body. A fluid, such as oil, may be supplied (Oil Supply) to the enclosed space, and a vacuum may be supplied (Vacuum Source) to the enclosed space.

FIG. 9A illustrates a system 900A combining the features of vacuum and fluid injection. FIG. 9A is similar to FIG. 9, with some differences. A transducer 902A may comprise a hole 923, which may be used for placement of the transducer. A membrane 920 has an annular lip as described hereinabove (refer to 440, 740) to form an enclosed space (refer to 431, 731) between the membrane and a surface of the subject body. Bottom surface 924 of membrane 920 may be coated with a “hygiene membrane” 924A, which may be disposed and/or replaced after each treatment. “Hygiene membrane” 924A may include one or more holes (such as holes 925 and 926). The vacuum created may suck, through the holes, hygiene membrane” 924A and the body tissue. The hygiene membrane may comprise silicone, polyethylene or any other appropriate material.

In this manner, a combination treatment may be provided comprising vacuum massage and HIFU (high intensity focusing ultrasound), and ultrasonically-assisted drug delivery.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

What is claimed is:

1. Apparatus for lysing of adipose tissue comprising:
a transducer adapted to transmit ultrasound to a target area tissue of a subject body; and
a vacuum system adapted to suck a surface of the target area tissue into the transducer to facilitate acoustic contact between the surface of the target area tissue and the transducer.

2. The apparatus of claim 1, wherein said vacuum system is adapted to operate in a continuous mode.

3. The apparatus of claim 1, wherein said vacuum system is adapted to operate in a pulsed mode.

4. The apparatus of claim 1, wherein said vacuum system and said transducer are adapted to operate in a pulsed mode.

5. The apparatus of claim 1, wherein said vacuum system is adapted to operate in a pulsed mode to produce vacuum pulsation and said transducer is adapted to operate in a pulsed mode to produce ultrasonic pulsation, wherein the vacuum pulsation and the ultrasonic pulsation are adapted to be synchronized.

6. The apparatus of claim 1, wherein said vacuum system comprises a vacuum pump.

7. The apparatus of claim 1, further comprising a coupling element for enhancing acoustic coupling of the ultrasound to target area tissue of a subject body.

8. The apparatus of claim 7, wherein the coupling element comprises a membrane disposed between the transducer and the surface of the target area tissue of the subject body.

9. The apparatus of claim 8, wherein said membrane comprises an annular lip protruding from a peripheral region of a bottom surface of the membrane, defining an enclosed space between the bottom surface of the membrane and a surface of the subject body.

10. The apparatus of claim 9, wherein the annular lip has a substantially hemispherical profile.

11. The apparatus of claim 9, wherein the annular lip has a substantially rectangular profile.

12. The apparatus of claim 9, wherein the annular lip has a substantially U-shaped profile.

13. The apparatus of claim 9, wherein the annular lip comprises at least one passageway to allow vacuum suction of the surface of the target area tissue into the transducer.

14. The apparatus of claim 1, adapted to receive a replicable hygiene membrane.

15. The apparatus of claim 1, adapted to vacuum-clamp a replicable hygiene membrane.

16. The apparatus of claim 1, further comprising a fluid supplying element for supplying a fluid, through the transducer, to a contact area on the surface of the target area tissue of the subject body.

17. Method for lysing of adipose tissue comprising:
providing an ultrasonic transducer;
applying vacuum to suck a surface of a target area tissue of a subject body into the transducer to facilitate acoustic contact between the surface of the target area tissue and the transducer; and
transmitting ultrasound by the ultrasonic transducer to the target area tissue.

18. The method of claim 17, wherein applying vacuum comprises applying vacuum in a continuous mode.

19. The method of claim 17, further comprising applying vacuum in a pulsed mode.

20. The method of claim 17, wherein transmitting ultrasound comprises transmitting ultrasound in a pulsed mode.

21. The method of claim 17, further comprising applying vacuum in a pulsed mode to produce vacuum pulsation, wherein transmitting ultrasound comprises transmitting ultrasound in a pulsed mode to produce ultrasound pulsation and wherein the vacuum pulsation and the ultrasonic pulsation are adapted to be synchronized.

22. The method of claim 17, wherein the transducer is capable of producing ultrasonic waves with an intensity sufficient for inducing cavitational lysis of said adipose tissue.

23. The method of claim 17, wherein the transducer is capable of producing ultrasonic waves with an intensity sufficient for inducing thermal lysis of said adipose tissue.

24. The method of claim 17, wherein the transducer is actuated to transmit ultrasonic waves of sufficient intensity to cause lysis of said adipose tissue essentially without damaging adjacent non-adipose tissue.

25. The method of claim 17, further comprising supplying fluid through the transducer, to a contact area on the surface of the target area tissue of the subject body.

26. The method of claim 25, wherein supplying fluid comprises injecting the fluid.

27. The method of claim 25, wherein supplying fluid comprises vacuum-sucking the fluid.

28. System for lysing of adipose tissue comprising:
a transducer adapted to transmit ultrasound to a target area tissue of a subject body;
a vacuum system adapted to suck a surface of the target area tissue into the transducer to facilitate acoustic contact between the surface of the target area tissue and the transducer; and
a controller adapted to synchronize the actuation of said transducer and said vacuum system.
